(19) United States
(12) Patent Application Publication
(10) Pub. No.: US 2024/0238298 A1
(43) Pub. Date: Jul. 18, 2024

(54) JAK Inhibitors for Managing Conditions in Patients with Down's Syndrome or Other Trisomy

(71) Applicant: Emory University, Atlanta, GA (US)

(72) Inventors: Christina Gavegnano, Atlanta, GA (US); Susan Pereira Ribeiro, Atlanta, GA (US)

(21) Appl. No.: 18/561,673

(22) PCT Filed: May 26, 2022
(86) PCT No.: PCT/US2022/031046

§ 371 Date: Nov. 16, 2023

Related U.S. Application Data
(60) Provisional application No. 63/193,279, filed on May 26, 2021.

(51) Int. Cl. A61K 31/519; A61K 39/00; A61K 39/39

(52) U.S. Cl. CPC A61K 31/519; A61K 39/39; A61K 2039/55511

(57) ABSTRACT

This disclosure relates to uses of JAK inhibitors for treating Down's Syndrome and other conditions associated with trisomy or preventing or reversing the deterioration of conditions or symptoms associated therewith. In certain embodiments, this disclosure relates to methods of treating Down's Syndrome or other trisomy comprising administering an effective amount of a JAK Inhibitor to a subject in need thereof.

### CROSS-REFERENCE TO RELATED APPLICATIONS

This application claims the benefit of U.S. Provisional Application No. 63/193,279 filed May 26, 2021. The entirety of this application is hereby incorporated by reference for all purposes.

### BACKGROUND

Down's Syndrome, the most frequent cause of genetically defined intellectual disability, is caused by the presence of an extra copy of the chromosome 21 (trisomy 21). Down's Syndrome patients are prone to certain birth defects, facial features, heart defects, and learning problems. Interventions early in life often attempt to improve their physical and intellectual abilities. Services include speech, occupational, and physical therapy. Therapeutic treatments are not directed to the biological mechanisms of Down's Syndrome. Thus, there is an unmet medical need to identify improved therapies.

The 21^{st} chromosome encodes inflammatory factors, including interferons, which are governed by the JAK-STAT signaling cascade. Lee et al. report the potential role of JAK-STAT signaling pathway in the neurogenic-to-gliogenic shift in Down's Syndrome brain. Neural Plasticity, 2016, Article ID 7434191.

Prasher et al. report donepezil, rivastigmine, galantamine, and memantine for the treatment of dementia in Alzheimer's disease in adults with Down Syndrome. Int J Geriatr Psychiatry, 2004, 19:509-515.

Furumoto et al. report JAK inhibitors for advancing the treatment of immune and hematologic disorders. BioDrugs, 2013, 27(5):431-438.

Assadiasl et al. report baricitinib is a JAK1/2 inhibitor for treating moderate to severe rheumatoid arthritis (RA) and control of exaggerated inflammatory responses. The Journal of Clinical Pharmacology, 2021, 61(10): 1274-1285.

References cited herein are not an admission of prior art.

### SUMMARY

This disclosure relates to uses of JAK inhibitors for treating Down's Syndrome and other conditions associated with trisomy or preventing or reversing the deterioration of conditions or symptoms associated therewith. In certain embodiments, this disclosure relates to methods of treating Down's Syndrome or other trisomy comprising administering an effective amount of a JAK Inhibitor to a subject in need thereof.

In certain embodiments, this disclosure relates to methods of treating or preventing Down's Syndrome using Jak inhibitors as a treatment, prevention, or reversal of disease in people living with Down's Syndrome or other trisomy.

In certain embodiments, the JAK inhibitor is baricitinib. In certain embodiments, the JAK inhibitor, e.g., baricitinib, tofacitinib, upadacitinib, etc., inhibits JAK1 and/or JAK2, JAK3 or TYK2 kinase activity thereby interfering with the cytokine-mediated signaling through JAK1 and/or JAK2 phosphorylation.

In certain embodiments, administering the JAK inhibitor e.g., baricitinib, tofacitinib, upadacitinib, etc., is used to treat or prevent or reverse specific trisomy-21-driven neurological dysregulation in patients with Down's Syndrome or other trisomy.

In certain embodiments, the administering the JAK inhibitor e.g., baricitinib, tofacitinib, upadacitinib, etc., is used to treat or prevent or reverse trisomy-driven cognitive decline and existing cognitive deficits in patients with Down's Syndrome or other trisomy.

In certain embodiments, administering the JAK inhibitor e.g., baricitinib, tofacitinib, upadacitinib, etc., is used to treat or prevent or reverse trisomy-21-driven dysregulation that confers unique comorbidities such as cardiovascular, diabetic, pancreatic, malignancies, and neurological comorbidities in patients with Down's Syndrome or other trisomy.

In certain embodiments, administering the JAK inhibitor e.g., baricitinib, tofacitinib, upadacitinib, etc., is used to treat or prevent or reverse trisomy-21-driven dysregulation of lymphocytes and lymphocyte driven malignancies and other cancers or hematological cancers and/or infections in patients with Down's Syndrome or other trisomy.

In certain embodiments, administering the JAK inhibitor e.g., baricitinib, tofacitinib, upadacitinib, etc., is used to improved vaccine immune responsiveness by improvement of lymphocyte activation and inflammation dysregulation that is caused by trisomy 21; coadministration with vaccinations to improve vaccine responsiveness, e.g., reversal of lymphocyte and peripheral blood mononuclear cell restoration of immune dysregulation in patients with Down's Syndrome or other trisomy.

### BRIEF DESCRIPTION OF THE SEVERAL VIEWS OF THE DRAWINGS

FIGS. 1A-1B show data of in vitro modes of Down's Syndrome-like immune dysregulation in primary human microglia-like cells and primary human monocytes. The data indicates baricitinib reverses the effect of this unique phenotype as it relates to cognitive and CNS dysregulation. Reversal of lymphocyte and peripheral blood mononuclear cell restoration of immune dysregulation is observed in an in vitro model.

FIG. 1A shows in vitro data indicating baricitinib reduces microglia activation an immune dysregulation marker which is a hallmark of immune dysregulation in people living with Down's Syndrome.

FIG. 1B shows in vitro data indicating baricitinib reduces peripheral and CNS-trafficking monocyte activation an immune dysregulation marker which is a hallmark of immune dysregulation in people living with Down's Syndrome.

FIGS. 2A-2G show data indicating baricitinib reverses baseline inflammation unique to Down's Syndrome individuals associated with cognitive defects. Peripheral blood mononuclear cells from individuals with Down's Syndrome were treated with baricitinib (1 μM) for 24 hours and/or post-treatment with TCR or LPS, to activate adaptive cells or innate cells respectively. Markers of monocyte inflammation and cytokines and chemokines associated with cognitive impairment were measured. The data indicates that baricitinib reverses markers of basal level inflammation. UNS denotes Down's Syndrome patient cells that are not stimulated (unstimulated).

FIG. 2A shows data for IFI16MFI monocytes.

FIG. 2B shows data for NLRP3MFI monocytes.

FIG. 2C shows data for CD101+18h LPS

FIG. 2D shows data for ID2+18h LPS

FIG. 2E shows data for IL18.

FIG. 2F shows data for MCP-1.

FIG. 2G shows data for MIP1-a+18h TCR.

FIGS. 3A-3E show data indicating baricitinib reverses the unique baseline inflammation of peripheral immune cells from Down's Syndrome individuals associated with several comorbidities unique to Down's Syndrome. Peripheral blood mononuclear cells from individuals with Down's Syndrome were treated with baricitinib (1 μM) for 24 hours. Markers of natural killer cell (NK) and inflammation, the capacity to secrete IL1b by CD4 T cells and other markers for inflammation were measured. The data indicates baricitinib reverses markers of basal level inflammation. UNS denotes Down's Syndrome patient cells that are not stimulated (unstimulated).

FIG. 3A shows data for CD16pCD56p NK Median (IFI-16).

FIG. 3B shows data for CD4p/IL1 bp.

FIG. 3C shows data for CD3p/CD4p Median (ASC).

FIG. 3D shows data for CD3p/CD4p Median (IFI-16).

FIG. 3E shows data for CD3p/CD4p Median (NLRP3).

FIG. 4A-4C show data indicating baricitinib reverses baseline immune exhaustion due to trisomy 21 rescuing the ability to fight infection and malignancies that is impaired in Down's Syndrome. This indicates baricitinib can reduce elevated immune exhaustion marker PD1 levels in T cells and related markers. Peripheral blood mononuclear cells from individuals with Down's Syndrome were treated with baricitinib (1 μM) for 24 hours and/or were exposed to the inflammatory stimulant TCR. Baricitinib reduced PD1 and TCF1 expression on T cells. UNS denotes Down's Syndrome patient cells that are not stimulated (unstimulated).

FIG. 4A shows data on markers of T cells exhaustion such as PD1 in CD4 T cells from Down's Syndrome patients.

FIG. 4B shows data on CD8 T cells from Down's Syndrome patients.

FIG. 4C shows data on T cell stemness TCF-1 in cells from Down's Syndrome patients.

FIGS. 5A and 5B shows data indicating baricitinib decreases markers of B cell inflammation unique to Down's Syndrome, in cells from Down's Syndrome Patients. Peripheral blood mononuclear cells from individuals with Down's Syndrome were treated with baricitinib (1 μM) for 24 hours. UNS denotes Down's Syndrome patient cells that are not stimulated (unstimulated).

FIG. 5A shows data indicating baricitinib reduced marker (IFI-16) in B cell inflammation.

FIG. 5B shows data indicating baricitinib reduced marker (NLRP3) in B cell inflammation.

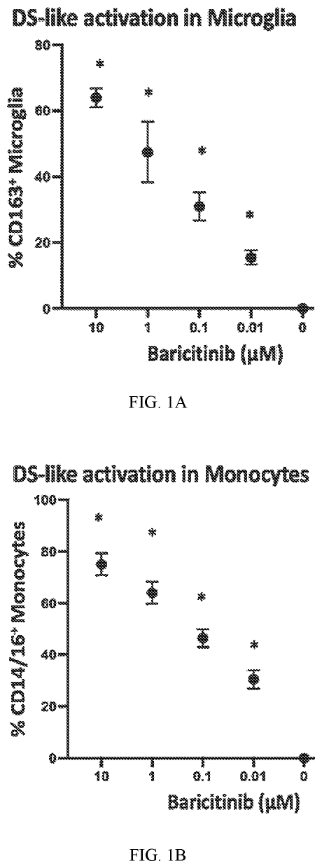

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited.

An “embodiment” of this disclosure refers to an example, but not necessarily limited to such example. As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

It must be noted that, as used in the specification and the appended claims, the singular forms “a,” “an,” and “the” include plural referents unless the context clearly dictates otherwise. In this specification and in the claims that follow reference will be made to a number of terms that shall be defined to have the following meanings unless a contrary intention is apparent.

As used in this disclosure and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) have the meaning ascribed to them in U.S. Patent law in that they are inclusive or open-ended and do not exclude additional, unrecited elements or method steps.

“Consisting essentially of” or “consists of” or the like, when applied to methods and compositions encompassed by the present disclosure refers to compositions like those disclosed herein that exclude certain prior art elements to provide an inventive feature of a claim, but which may contain additional composition components or method steps, etc., that do not materially affect the basic and novel characteristic(s) of the compositions or methods.

The terms “JAK inhibitor” or “Janus kinase inhibitor” refers to any variety of compounds, (small molecules, biological molecules, anti-JAK antibodies, interfering RNA, shRNA, nucleic acids/vectors encoding proteins, CRISPR-Cas mediated systems, etc.) that, when in contact with a Janus kinase enzyme (JAK1, JAK2, JAK3, TYK2) specifically bind with a Janus kinase enzyme altering the JAK-STAT signaling pathway. Examples include baricitinib, tofacitinib, upadacitinib cerdulatinib, gandotinib, lestaurtinib, momelotinib, pacritinib ruxolitinib, oclacitinib, peficitinib, fedratinib, filgotinib, delgocitinib, abrocitinib, all of which are contemplated for uses disclosed herein.

As used herein, the term “small molecule” refers to any variety of covalently bound molecules with a molecular weight of less than 900 or 1000 Da. Typically, the majority of atoms include carbon, hydrogen, oxygen, nitrogen, and to a lesser extent sulfur and/or a halogen. Examples include steroids, short peptides, mono or polycyclic aromatic or non-aromatic, heterocyclic compounds.

As used herein, “subject” refers to any animal, preferably a human patient.

As used herein, the terms “treat” and “treating” are not limited to the case where the subject (e.g. patient) is cured and the disease is eradicated. Rather, embodiments, of the present disclosure also contemplate treatment that merely reduces symptoms, reverses, and/or delays disease progression.

As used herein, the terms “prevent” and “preventing” include the prevention of the recurrence, spread or onset. It is not intended that the present disclosure be limited to complete prevention. In some embodiments, the onset is delayed, or the severity of the disease is reduced.

As used herein, the term “combination with” when used to describe administration of an agent with an additional treatment means such that the agent may be administered prior to, together with, or after the additional treatment, or a combination thereof, such that multiple agents are available at some overlapping time.

The term “effective amount” refers to that amount of a compound or pharmaceutical composition described herein that is sufficient to effect the intended application including, but not limited to, disease treatment, as illustrated below. In relation to a combination therapy, an “effective amount” indicates the combination of agent results in synergistic or additive effect when compared to the agents individually. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The specific dose will vary depending on, for example, the particular compounds chosen, the dosing regimen to be followed, whether it is administered in combination with other agents, timing of administration, the tissue to which it is administered, and the physical delivery system in which it is carried.

The terms “drug,” “agent,” “pharmaceutical agent,” and similar terms are used interchangeably herein, and mean and include an agent, drug, compound, composition of matter or mixture thereof, including its formulation, which provides some therapeutic, often beneficial, effect. This includes any physiologically or pharmacologically active substance that produces a localized or systemic effect. Examples include chemotherapy agents, analgesics, steroidal anti-inflammatories, non-steroidal anti-inflammatories, statins, antibiotics, anti-bacterial agents, anti-neoplastics, anti-spasmodics, modulators of cell-extracellular matrix interactions, proteins, hormones, enzymes and enzyme inhibitors, anticoagulants and/or antithrombic agents, and vasodilating agents.

The terms “anti-inflammatory” and “anti-inflammatory agent” are also used interchangeably herein and mean and include a “pharmacological agent” and/or “active agent formulation”, which, when a therapeutically effective amount is present the drug prevents or treats bodily tissue inflammation i.e., the protective tissue response to injury or destruction of tissues, which serves to destroy, dilute, or wall off both the injurious agent and the injured tissues. Examples include alclofenac, alclometasone dipropionate, alpha amylase, amcinafal, amfenac sodium, anakinra, anirolac, balsalazide disodium, bendazac, benoxaprofen, bromelains, broperamole, budesonide, carprofen, cliprofen, clobetasol propionate, clobetasone butyrate, clopirac, cortodoxone, decanoate, deflazacort, depo-testosterone, desonide, desoximetasone, dexamethasone dipropionate, diclofenac potassium, diclofenac sodium, diflorasone diacetate, diflumidone sodium, diflunisal, difluprednate, dimethyl sulfoxide, enolicam sodium, etodolac, felbinac, fenamole, fenbufen, fenclofenac, fendosal, fenpipalone, fentiazac, flazalone, flufenamic acid, flunisolide acetate, flunixin, flunixin meglumine, fluorometholone acetate, flurbiprofen, fluticasone propionate, furaprofen, halcinonide, halobetasol propionate, ibuprofen, ibuprofen aluminum, ibuprofen piconol, indomethacin, indomethacin sodium, indoprofen, isoxepac, isoxicam, ketoprofen, lofemizole hydrochloride, loteprednol etabonate, meclofenamate sodium, meclofenamic acid, mefenamic acid, mesalamine, methenolone, methenolone acetate, nabumetone, nandrolone, naproxen, naproxen sodium, naproxol, olsalazine sodium, oxaprozin, oxyphenbutazone, oxymetholone, pirfenidone, piroxicam, piroxicam cinnamate, piroxicam olamine, pirprofen, proquazone, proxazole, proxazole citrate, salsalate, stanozolol, sudoxicam, sulindac, suprofen, talniflumate, tenidap, tenidap sodium, tenoxicam, testosterone, testosterone blends, tiopinac, tixocortol pivalate, tolmetin, tolmetin sodium, triclonide, triflumidate, zidometacin, and zomepirac sodium.

A “chemotherapy agent,” “chemotherapeutic,” “anti-cancer agent,” or the like, refer to molecules that are recognized to aid in the treatment of a cancer. Contemplated examples include the following molecules or derivatives such as abemaciclib, abiraterone acetate, methotrexate, paclitaxel, adriamycin, acalabrutinib, brentuximab vedotin, ado-trastuzumab emtansine, aflibercept, afatinib, netupitant, palonosetron, imiquimod, aldesleukin, alectinib, alemtuzumab, pemetrexed disodium, copanlisib, melphalan, brigatinib, chlorambucil, amifostine, aminolevulinic acid, anastrozole, apalutamide, aprepitant, pamidronate disodium, exemestane, nelarabine, arsenic trioxide, ofatumumab, atezolizumab, bevacizumab, avelumab, axicabtagene ciloleucel, axitinib, azacitidine, carmustine, belinostat, bendamustine, inotuzumab ozogamicin, bevacizumab, bexarotene, bicalutamide, bleomycin, blinatumomab, bortezomib, bosutinib, brentuximab vedotin, brigatinib, busulfan, irinotecan, capecitabine, fluorouracil, carboplatin, carfilzomib, ceritinib, daunorubicin, cetuximab, cisplatin, cladribine, cyclophosphamide, clofarabine, cobimetinib, cabozantinib-S-malate, dactinomycin, crizotinib, ifosfamide, ramucirumab, cytarabine, dabrafenib, dacarbazine, decitabine, daratumumab, dasatinib, defibrotide, degarelix, denileukin diftitox, denosumab, dexamethasone, dexrazoxane, dinutuximab, docetaxel, doxorubicin, durvalumab, rasburicase, epirubicin, elotuzumab, oxaliplatin, eltrombopag olamine, enasidenib, enzalutamide, eribulin, vismodegib, erlotinib, etoposide, everolimus, raloxifene, toremifene, panobinostat, fulvestrant, letrozole, filgrastim, fludarabine, flutamide, pralatrexate, obinutuzumab, gefitinib, gemcitabine, gemtuzumab ozogamicin, glucarpidase, goserelin, propranolol, trastuzumab, topotecan, palbociclib, ibritumomab tiuxetan, ibrutinib, ponatinib, idarubicin, idelalisib, imatinib, talimogene laherparepvec, ipilimumab, romidepsin, ixabepilone, ixazomib, ruxolitinib, cabazitaxel, palifermin, pembrolizumab, ribociclib, tisagenlecleucel, lanreotide, lapatinib, olaratumab, lenalidomide, lenvatinib, leucovorin, leuprolide, lomustine, trifluridine, olaparib, vincristine, procarbazine, mechlorethamine, megestrol, trametinib, temozolomide, methylnaltrexone bromide, midostaurin, mitomycin C, mitoxantrone, plerixafor, vinorelbine, necitumumab, neratinib, sorafenib, nilutamide, nilotinib, niraparib, nivolumab, tamoxifen, romiplostim, sonidegib, omacetaxine, pegaspargase, ondansetron, osimertinib, panitumumab, pazopanib, interferon alfa-2b, pertuzumab, pomalidomide, mercaptopurine, regorafenib, rituximab, rolapitant, rucaparib, siltuximab, sunitinib, thioguanine, temsirolimus, thalidomide, thiotepa, trabectedin, valrubicin, vandetanib, vinblastine, vemurafenib, vorinostat, zoledronic acid, or combinations thereof such as cyclophosphamide, methotrexate, 5-fluorouracil (CMF); doxorubicin, cyclophosphamide (AC); mustine, vincristine, procarbazine, prednisolone (MOPP); sdriamycin, bleomycin, vinblastine, dacarbazine (ABVD); cyclophosphamide, doxorubicin, vincristine, prednisolone (CHOP); bleomycin, etoposide, cisplatin (BEP); epirubicin, cisplatin, 5-fluorouracil (ECF); epirubicin, cisplatin, capecitabine (ECX); methotrexate, vincristine, doxorubicin, cisplatin (MVAC).

“Cancer” refers any of various cellular diseases with malignant neoplasms characterized by the proliferation of cells. Cancer may or may not be present as a tumor mass with a defined boundary. It is not intended that the diseased cells must actually invade surrounding tissue and metastasize to new body sites. Cancer can involve any tissue of the body and have many different forms in each body area. Within the context of certain embodiments, whether “cancer is reduced” may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, HER2 for breast cancer, or others. Contemplated cancers include hematological cancers leukemia, lymphoma, and multiple myeloma, breast cancer, non-small cell lung cancer and small cell lung cancer, bronchus cancer, prostate cancer, colon cancer, rectum cancer, skin cancer, melanoma, bladder cancer, non-Hodgkin lymphoma, kidney cancer, renal cancer, pelvis cancer, endometrial cancer, pancreatic cancer, thyroid cancer, and liver cancer.

As used herein, a “chimeric antigen receptor” or “CAR” refers to a protein receptor, which introduces an antigen specificity, via an antigen binding domain, onto immune cells to which it is expressed (for example natural kill or T cells such as naive T cells, central memory T cells, effector memory T cells or combination thereof) thus combining the antigen binding properties of the antigen binding domain with the T cell activity (e.g. lytic capacity and self-renewal) of T cells or other immune cells. A CAR typically includes an extracellular antigen-binding domain (ectodomain), a transmembrane domain and an intracellular signaling domain. The intracellular signaling domain generally contains at least one immunoreceptor tyrosine-based activation motif (ITAM) signaling domain, e.g. derived from CD3zeta, and optionally at least one costimulatory signaling domain, e.g. derived from CD28 or 4-1BB.

In order to improve the ability of immune cells to kill cancerous cells, T cells or other immune cells can be isolated from the blood of a patient and genetically altered to express chimeric antigen receptors (CARs) to specifically target proteins expressed on the surface of cancerous cells and stimulate an immune response. When put back into the patient, the cells attack the cancerous cells. Brentjens et al. report that T cells altered to bind CD19 can induce remissions of cancer in adults with chemotherapy-refractory acute lymphoblastic leukemia. Sci Transl Med, 2013, 5(177): 177ra38.

The term “nucleic acid” refers to a polymer of nucleotides, or a polynucleotide, e.g., RNA, DNA, or a combination thereof. The term is used to designate a single molecule, or a collection of molecules. Nucleic acids may be single stranded or double stranded and may include coding regions and regions of various control elements.

The term “encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (e.g., rRNA, tRNA and mRNA) or a defined sequence of amino acids and the biological properties resulting therefrom. Thus, a gene, cDNA, or RNA, encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The terms “polypeptide,” “peptide,” and “protein” are used interchangeably herein to refer to polymers of amino acids of any length. The polymer can comprise modified amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component.

### Methods of Use

This disclosure relates to uses of JAK inhibitors for treating Down's Syndrome and other conditions associated with a trisomy or reversing or preventing the deterioration of conditions or symptoms associated therewith. In certain embodiments, this disclosure relates to methods of treating or preventing Down's Syndrome or associated conditions comprising administering an effective amount of a JAK Inhibitor to a subject in need thereof.

In certain embodiments, this disclosure relates to methods of treating Down's Syndrome comprising administering an effective amount of a JAK inhibitor to a patient diagnosed with Down's Syndrome. In certain embodiments, the JAK inhibitor is baricitinib. In certain embodiments, the treatment improves memory. In certain embodiments, the treatment improves cognitive function. In certain embodiments, the treatment improves speech. In certain embodiments, the treatment improves motor skills. In certain embodiments, the treatment reduces dementia. In certain embodiments, the treatment improves cardiovascular health or reduces the risk of coronary artery disease, reduces plaque, or reduces the occurrence an adverse cardiovascular event such as a heart attack. In certain embodiments, the treatment reduces the loss of hearing and/or vision.

In certain embodiments, this disclosure relates to methods disclosed herein directed to the treatment or reversal of unique trisomy-21-induced comorbidities, treatment or reversal of unique trisomy-21-cognitive deficits and decline, and co-administration with vaccinations and other therapies (such as cellular therapies, antibodies therapies, small molecules therapies, etc.) to mitigate poor vaccine or therapy responsiveness conferred by the unique trisomy-driven peripheral blood mononuclear cell and lymphocyte dysregulation.

In certain embodiments, this disclosure relates to methods disclosed herein directed to the treatment or reversal of trisomy-21-driven dysregulation that confers unique comorbidities in Down's Syndrome individuals, across cardiovascular, diabetic (type I or type II), pancreatic, malignancies, and/or neurological comorbidities.

In certain embodiments, this disclosure relates to methods disclosed herein directed to the treatment or reversal of unique trisomy-21-driven dysregulation of lymphocytes and lymphocyte driven malignancies and related disorders and infections.

In certain embodiments, this disclosure relates to methods disclosed herein directed to improved vaccine responsiveness and responsiveness of other therapies (such as cellular, antibodies, small molecules, molecular, etc.) by improvement of lymphocyte homeostasis, activation and inflammation dysregulation that is caused by a trisomy, e.g., trisomy 21. In certain embodiments, this disclosure relates to methods disclosed herein directed to pre-administration and/or co-administration with (in combination with) vaccinations or other therapies (anti-inflammatory agents, chemotherapy agents).

In certain embodiments, the trisomy is trisomy 21 (Down's Syndrome). Also contemplated are subjects with trisomy 18 (Edward's syndrome) or trisomy 13 (Patau syndrome), Klinefelter syndrome, and triple x syndrome.

In certain embodiments, the JAK inhibitor is baricitinib. In certain embodiments, the JAK inhibitor is tofacitinib. In certain embodiments, the JAK inhibitor is upadacitinib. In certain embodiments, the JAK inhibitor, e.g., baricitinib, tofacitinib, upadacitinib, etc., inhibits JAK1 and/or JAK2 kinase activity thereby interfering with the cytokine-mediated signaling through JAK1 and/or JAK2, and/or JAK3 and/or TYK2 phosphorylation. In certain embodiments, the JAK inhibitor is selected from baricitinib, tofacitinib, upadacitinib cerdulatinib, gandotinib, lestaurtinib, momelotinib, pacritinib ruxolitinib, oclacitinib, peficitinib, fedratinib, filgotinib, delgocitinib, and abrocitinib.

In certain embodiments, the subject is a human subject, wherein the subject is less than 10 years old, the subject is less than 5 years old, or the subject is 10 years old or more than 10 years old. In certain embodiments, the subject is diagnosed with such a disease or condition, before or after birth.

In certain embodiments, administering the JAK inhibitor e.g., baricitinib, tofacitinib, upadacitinib, etc., is used to treat or prevent or reverse specific trisomy-21-driven neurological dysregulation in patients with Down's Syndrome or other trisomy.

In certain embodiments, administering the JAK inhibitor e.g., baricitinib, tofacitinib, upadacitinib, etc., is used to treat or prevent or reverse trisomy-driven cognitive decline and existing cognitive deficits in patients with Down's Syndrome or other trisomy.

In certain embodiments, administering the JAK inhibitor e.g., baricitinib, tofacitinib, upadacitinib, etc., is used to treat or prevent or reverse trisomy-21-driven dysregulation that confers unique comorbidities, such as cardiovascular, diabetic (type I or type II diabetes), pancreatic, malignancies, and neurological comorbidities in patients with Down's Syndrome or other trisomy. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent the development or exaggeration of a physical conditions or birth defect, e.g. abnormal bone growth, abnormal facial features, brachycephaly (skull deformity characterized by a shortened anteroposterior skull length and a widened biparietal diameter), palpebral fissures, low-set ears, mouth breathing, atlantoaxial instability, displacement of the tongue, excess skin on the back of the neck, flaccid muscles, muscle weaknesses, spinal curvatures, seborrheic dermatitis, single line on palm, thickening of the skin of the palms and soles. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent the deterioration of vision, lazy eye, eye spots, deterioration of hearing, hearing loss. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent the development of thyroid disease, heart defects, heart disease, e.g., congenital heart disease. In certain embodiments, administering the JAK inhibitor e.g., baricitinib, tofacitinib, upadacitinib, etc., treats or prevents coronary artery disease (CAD). In certain embodiments, administering the JAK inhibitor e.g., baricitinib, tofacitinib, upadacitinib, etc., treats or prevents hypothyroidism. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent intellectual disability or developmental delays, such as Down's Syndrome associated dementia, learning disability, speech delay, abnormal difficulty thinking, or to improve memory and understanding. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent gastrointestinal malformations such as duodenal stenosis and Hirschsprung disease. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent infections in the upper respiratory tract. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent Celiac disease and other food intolerances. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent seizure disorder. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent obesity, dental problems, and apnea/obstructive sleep apnea. In certain embodiments the subject is diagnosed with such a condition.

In certain embodiments, administering the JAK inhibitor e.g., baricitinib, tofacitinib, upadacitinib, etc., is used in patients with Down's Syndrome or other trisomy to treat or prevent polycythemia. In certain embodiments the subject is diagnosed with such a condition.

In certain embodiments, this disclosure relates to methods using of a JAK inhibitor such as baricitinib to treat, prevent, or reverse disease in combination with other drugs such as rivastigmine, galantamine, memantine, and donepezil. In certain embodiments, the subject is diagnosed with Down's Syndrome associated dementia (DSAD). In certain embodiments the subject is diagnosed with such a condition.

In certain embodiments, administering the JAK inhibitor e.g., baricitinib, tofacitinib, upadacitinib, etc., treats or prevents recurrent skin and soft tissue infections. In certain embodiments the subject is diagnosed with such a condition.

In certain embodiments, the administering the JAK inhibitor e.g., baricitinib, tofacitinib, upadacitinib, etc., is used to treat or prevent or reverse trisomy-21-driven dysregulation of lymphocytes and lymphocyte driven malignancies, any cancer, and related disorders and infections in patients with Down's Syndrome or other trisomy. In certain embodiments, the JAK inhibitor is administered in combination with an anticancer agent or anti-inflammatory agent. In certain embodiments, the JAK inhibitor is administered in combination with a cell therapy such as CAR therapy. In certain embodiments, the JAK inhibitor is administered in combination with a hematopoietic progenitor cell transplantation, e.g., derived from bone marrow, circulating (peripheral) blood and/or umbilical cord blood. In certain embodiments, the JAK inhibitor is administered in combination with radiation therapy.

In certain embodiments, administering the JAK inhibitor e.g., baricitinib, tofacitinib, upadacitinib, etc., is used in patients with Down's Syndrome or other trisomy to treat or prevent cancer or a hematologic disorder, such as acute myeloid leukemia, acute lymphoblastic leukemia, and anemia. In certain embodiments the subject is diagnosed with such a condition. In certain embodiments, the JAK inhibitor is administered in combination with an anticancer agent or chimeric antigen receptor (CAR) therapy. In certain embodiments, the anticancer agent is a checkpoint inhibitor, an anti-PD-1, anti-PD-L1 anti-CTLA4 antibody or combinations thereof. In certain embodiments, the anti-CTLA4 antibody is ipilimumab or tremelimumab. In certain embodiments, the anti-PD1 antibody is nivolumab, pembrolizumab, or cemiplimab. In certain embodiments, the anti-PD-L1 antibody is atezolizumab, avelumab, or durvalumab.

In certain embodiments, the JAK inhibitor is administered to a subject with a lymphodepleted environment due to prior or concurrent administration of a lymphodepleting agent. In certain embodiments, the of lymphodepleting agent is cyclophosphamide, fludarabine, or combination thereof.

In certain embodiments, the hematological malignancy is selected from leukemia, acute lymphoblastic leukemia (ALL), acute myelogenous leukemia (AML), chronic lymphocytic leukemia (CLL), small lymphocytic lymphoma (SLL), chronic myelogenous leukemia, acute monocytic leukemia (AMOL), chronic myeloid leukemia (CML), B-cell acute lymphoblastic leukemia (B-ALL), myeloproliferative neoplasms (MPNs), and lymphomas, Hodgkin's lymphomas, and non-Hodgkin's lymphomas such as Burkitt lymphoma, B-cell lymphoma, or diffuse large B-cell lymphoma (DLBCL).

In certain embodiments, this disclosure relates to methods of treating cancer comprising administering an effective amount of a JAK inhibitor in combination with a chemotherapy agent and/or cell therapy to a patient diagnosed with Down's Syndrome. In certain embodiments, the JAK inhibitor is baricitinib. In certain embodiments, the cancer is a hematological cancer. In certain embodiments, the cancer is a leukemia. In certain embodiments, the cell therapy is a hematopoietic progenitor cell transplantation or chimeric antigen receptor T cell therapy.

In certain embodiments, administering the JAK inhibitor e.g., baricitinib, tofacitinib, upadacitinib, etc., is used to improved vaccine immune responsiveness by improvement of lymphocyte activation and inflammation dysregulation that is caused by trisomy 21; coadministration with vaccinations to improve vaccine responsiveness, e.g., reversal of lymphocyte and peripheral blood mononuclear cell restoration of immune dysregulation in patients with Down's Syndrome or other trisomy.

In certain embodiments, this disclosure relates to methods of improving effectiveness of a vaccine comprising administering an of a JAK inhibitor in combination with a vaccine to a patient diagnosed with Down's Syndrome. In certain embodiments, the JAK inhibitor is baricitinib. In certain embodiments, the vaccine is an influenza vaccine or coronavirus vaccine (COVID-19). In certain embodiments, the vaccine is a hepatitis A virus vaccine or hepatitis B virus vaccine. In certain embodiments, the vaccine is a human papillomavirus (HPV) vaccine. In certain embodiments, the vaccine is tetanus, diphtheria, pertussis, and/or meningococcal (meningitis) vaccine. In certain embodiments, the vaccine is a measles, mumps, and/or rubella vaccine. In certain embodiments, the vaccine is a varicella vaccine (Chickenpox), or a polio vaccine.

In certain embodiments, this disclosure relates to methods treating or preventing Down's Syndrome, or other trisomy or conditions related thereto as reported herein comprising administering an effective amount of a JAK Inhibitor to a pregnant mother wherein the fetus is diagnosed with a trisomy or at risk of a birth defect.

In certain embodiments, diagnosis of Down's Syndrome and other genetic tests are performed before a baby is born through amniocentesis or chorionic villus sampling (CVS). In certain embodiments, diagnosis begins at birth based on the physical appearance of the child after analyzing chromosomes. In certain embodiments, the fetus is at risk of Down's Syndrome or other birth defect due to a diagnosis of an increased abnormal nuchal fold thickness and/or nasal bone. In certain embodiments, the increased abnormal nuchal fold thickness and/or nasal bone thickness is identified by an ultrasound measurement. In certain embodiments, the diagnosis is at between 10 and 14 weeks of gestation or 13 weeks to 27 weeks and 6 days of gestation. In certain embodiments, birth defect is a heart, abdominal wall, and/or skeletal defect.

### Use of Jak Inhibitors for Treatment or Prevention of Trisomy-21 Conditions, Comorbidities, and Immune Dysregulation

The 21st chromosome is responsible for encoding inflammatory factors, including interferons, which are governed by the JAK-STAT signaling cascade. Individuals with Down's Syndrome (trisomy 21) therefore suffer from a significant amount of comorbidities that are driven by chronic, unchecked inflammation across compartments systemically, including the central nervous system (CNS). Together, this inflammation drives a uniquely dysregulated immune phenotype. Baricitinib is an orally bioavailable JAK1 and JAK2 selective inhibitor that is clinically approved for rheumatoid arthritis, atopic dermatitis, and hospitalized COVID-19 patients. Hallmarks of Down's Syndrome immune dysregulation include increase in activation across both peripheral blood mononuclear cells (lymphocytes, monocytes, megakaryocytes, neutrophils, and macrophages), and CNS cells including microglia and macrophage-like cells, specifically driven by the trisomy 21 phenotype unique in people living with Down's Syndrome.

It is contemplated that subjects at all ages with Down's Syndrome benefit from specific, potent, safe, orally bioavailable agents that target the unique immune dysregulation that is conferred by the trisomy 21 in Down's Syndrome individuals. It is contemplated that targeted blockade of the trisomy 21-induced inflammatory dysregulation mitigates cellular and inflammatory events that drive comorbidities in Down's Syndrome individuals, while simultaneously provide an opportunity to reverse the trisomy-drive chronic inflammation observed in the CNS compartment of Down's Syndrome individuals that causes increased cognitive deficits, early dementia and Alzheimer's like diseases, and overall neurodegeneration versus healthy, disomy 21 counterparts. It is contemplated that methods disclosed herein are able to ameliorate comorbidities directly associated with the heightened basal levels of inflammation or with its downstream side-effects, which are driven by the unique trisomy 21 in Down's Syndrome individuals. Also contemplated are methods to improve immune homeostasis and fight against infections, cancers and ameliorate responses to immune interventions (vaccines, cellular therapies, immune therapies in general).

In certain embodiments, this disclosure relates to the use of JAK inhibitors such as baricitinib as a treatment, prevention, or reversal of disease/comorbidities, and improve immune responses in people living with Down's Syndrome or other trisomy.

In certain embodiments, this disclosure relates to the use of JAK inhibitors, such as baricitinib, as a treatment, prevention, or reversal of disease in combination with other agents commonly contained in cocktails for people diagnosed with Down's Syndrome or other trisomy.

In certain embodiments, this disclosure relates to the use of JAK inhibitors such as baricitinib for co-administration with vaccinations, immune and cellular therapies to improve therapy responsiveness in people diagnosed with Down's Syndrome or other trisomy; such as poor vaccine responsiveness a known, unique trisomy-21-driven mechanism with no therapeutic intervention to improve or treat this condition.

In certain embodiments, this disclosure relates to the use of JAK inhibitors such as baricitinib for pre-treatment prior to vaccinations, immune and cellular therapies to improve therapy responsiveness in people diagnosed with Down's Syndrome or other trisomy; such as poor vaccine responsiveness a known, unique trisomy-21-driven mechanism with no therapeutic intervention to improve or treat this condition. In certain embodiments, pre-treatment is one, two, three, or more days before vaccination. In certain embodiments, pre-treatment is one, two, or more weeks before vaccination. In certain embodiments, pre-treatment is daily administration.

Hallmarks of Down's Syndrome immune dysregulation include increase in activation across both peripheral blood mononuclear cells (lymphocytes, monocytes, megakaryocytes, neutrophils, and macrophages), and CNS cells including microglia and macrophage-like cells, specifically driven by the trisomy 21 phenotype which is unique in people living with Down's Syndrome.

In vitro experiments reported herein indicate that physiologically relevant concentrations of baricitinib can block immune activation and dysregulatory events that mimic the unique dysregulation driven by trisomy 21 in people living with Down's Syndrome. Data was collected in macrophages, microglia, a mixed population of blood mononuclear cells (lymphocytes, monocytes, megakaryocytes, neutrophils, and macrophages), and CD4 T cells, which are known to 1) drive disease progression and comorbidity in people living with Down's Syndrome, and 2) drive poor immune responsiveness in people living with Down's Syndrome. These data indicate that baricitinib can be used for the treatment or reversal of multiple indications within the unique Down's Syndrome disease phenotype, as well as used to improve vaccine and other therapies responsiveness, that are poor due to lymphocyte dysregulation and activation.

Baricitinib Reverses Baseline Inflammation in Down's Syndrome Individuals Associated with Cognitive Deficits

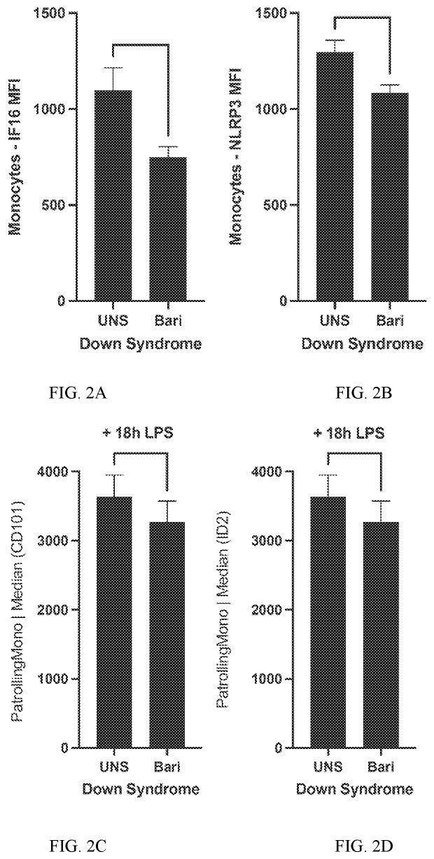

Experimental data indicates that baricitinib can reduce the baseline, elevated inflammation found in individuals with Down's Syndrome due to their trisomy 21 (FIGS. 2A-G). Immune dysregulation in myeloid cells and important cytokine/chemokines are responsible for brain inflammation and the development of early onset of severe cognitive deficits and neurological dysregulation pathologies such as Alzheimer's Disease in people with Down's Syndrome. These data demonstrate that baricitinib can be used as a therapeutic to treat existing brain and systemic inflammatory conditions that are the major morbidity in people with Down's Syndrome, and hence restore their inflammatory deregulatory milieu.

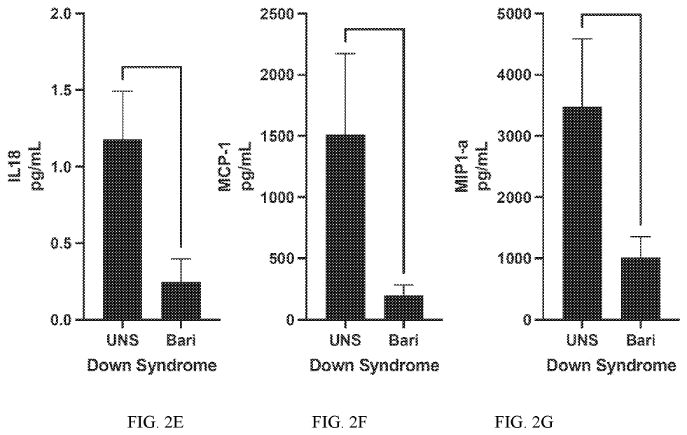

Baricitinib reduced NK inflammation, secretion if IL1b, and general inflammasome markers in immune cells (FIGS. 3A-E). This data indicates that baricitinib reverses unique baseline inflammation of peripheral immune cells from Down's Syndrome individuals associated with Baricitinib can reduce the baseline, elevated inflammation found in several comorbidities in peripheral immune cells unique to individuals with Down's Syndrome (due to their trisomy 21), including immune dysregulation peripherally that is a major driver of systemic comorbidities in people with Down's Syndrome, such as cardiovascular disease, diabetes, autoimmunity, hypertension, malignancies, neurocognitive deficits, and inflammatory-driven pathologies in people with Down's Syndrome, and other co-morbidities associated to the hyperinflammation observed in Down's Syndrome subjects.

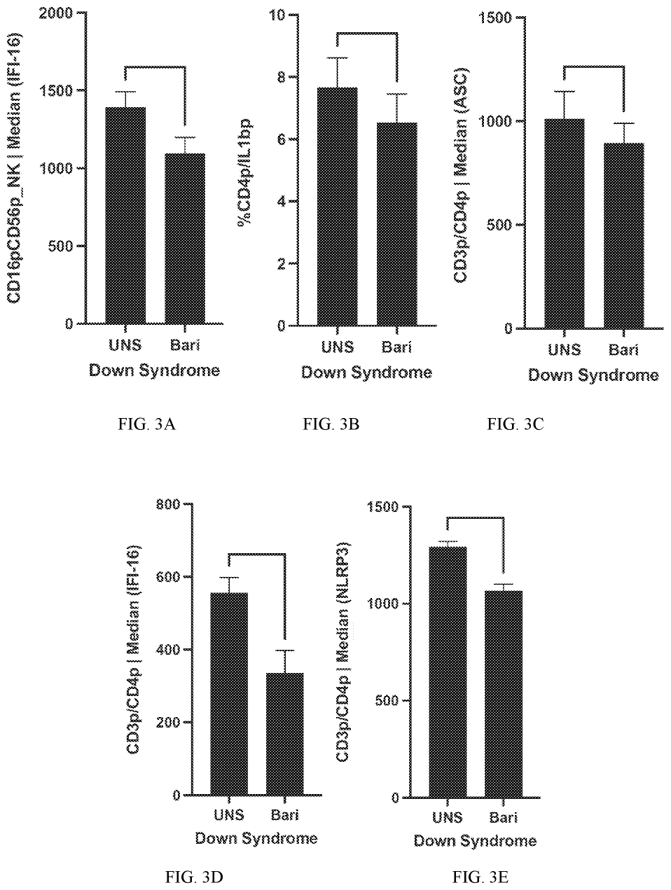

Baricitinib can reduce the baseline elevated immune exhaustion marker PD1 levels in T cells as well as markers of immune exhaustion and related markers (FIGS. 4A-C). This data indicates that baricitinib reverses baseline immune exhaustion caused by the unique trisomy 21, rescuing in ability to fight infection and malignancies that is impaired in Down's Syndrome. Reduction of these markers that are caused by the trisomy 21 allows the immune cells to function properly against viral infections and malignancies, which is otherwise impaired due to trisomy 21.

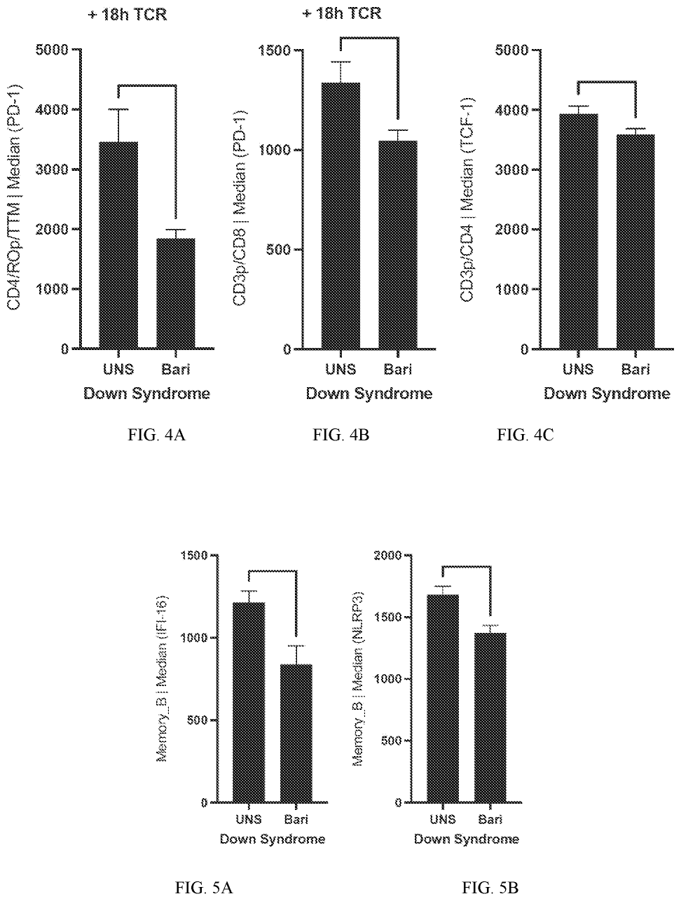

Data indicates that baricitinib reverses the inflammation of B cells unique to Down's Syndrome individuals (FIGS. 5A-B). Baricitinib reverses immune dysfunction that drives poor vaccine responsiveness that is uniquely found due to trisomy 21. B cells that are hyperactivated cannot respond to vaccines or other therapies; the unique inflammation caused by trisomy 21 that causes poor therapy responsiveness is prevented by baricitinib. This, along with better innate, CD4 and CD8 T cell responses, set the stage for a homeostatic environment and better response to therapies overall.

1. A method of treating Down's Syndrome comprising administering an effective amount of a JAK inhibitor to a patient diagnosed with Down's Syndrome.

2. The method of claim 1, wherein the JAK inhibitor is baricitinib.

3. The method of claim 1, wherein the treatment improves memory.

4. The method of claim 1, wherein the treatment improves speech.

5. The method of claim 1, wherein the treatment reduces dementia.

6. The method of claim 1, wherein the treatment improves cardiovascular health, reduces plaque, or reduces the risk of an adverse cardiovascular event.

7. The method of claim 1, wherein the treatment reduces the loss of hearing and/or vision.

8. A method of improving effectiveness of a vaccine comprising administering an of a JAK inhibitor in combination with a vaccine to a patient diagnosed with Down's Syndrome.

9. The method of claim 8, wherein the JAK inhibitor is baricitinib.

10. The method of claim 8, wherein the vaccine is an influenza vaccine or coronavirus vaccine.

11. The method of claim 8, wherein the vaccine is a hepatitis A virus vaccine or hepatitis B virus vaccine

12. The method of claim 8, wherein the vaccine is a human papillomavirus (HPV) vaccine.

13. The method of claim 8, wherein the vaccine is tetanus, diphtheria, pertussis, and/or meningococcal (meningitis) vaccine.

14. The method of claim 8, wherein the vaccine is a measles, mumps, and/or rubella vaccine.

15. The method of claim 8, wherein the vaccine is a varicella vaccine (Chickenpox), or a polio vaccine.

16. A method of treating cancer comprising administering an effective amount of a JAK inhibitor in combination with a chemotherapy agent and/or cell therapy to a patient diagnosed with Down's Syndrome.

17. The method of claim 16, wherein the JAK inhibitor is baricitinib.

18. The method of claim 16 wherein the cancer is a hematological cancer.

19. The method of claim 16 wherein the cancer is a leukemia.

20. The method of claim 16, wherein the cell therapy is a hematopoietic progenitor cell transplantation or chimeric antigen receptor T cell therapy.
